(19) United States
(12) Patent Application Publication
(10) Pub. No.: US 2024/0209300 A1
(43) Pub. Date: Jun. 27, 2024

(54) HIGH-THROUGHPUT MICROBIOREACTOR BASED PROCESS FOR EFFICIENT PRODUCTION OF LEVAN FROM MARINE BACILLUS SP

(71) Applicant: COUNCIL OF SCIENTIFIC AND INDUSTRIAL RESEARCH AN INDIAN REGISTERED BODY INCORPOR, New Delhi (IN)

(72) Inventors: Syed Gulam DASTAGER, Pune (IN); Vasudev Sahebrao WAGH, Pune (IN)

(21) Appl. No.: 18/166,862

(22) Filed: Feb. 9, 2023

(30) Foreign Application Priority Data
Dec. 27, 2022 (IN) 202211075840

(51) Int. Cl. C12M 1/00; C12N 1/20

(52) U.S. Cl. CPC C12M 23/02; C12N 1/20; C12N 2500/34

(57) ABSTRACT

The present invention relates to a process for the production of levan essentially free of contaminating polysaccharides using a bioreactor. More particularly, the present invention relates to a process for the increased production of levan using a strain of marine Bacillus sp. SGD-03. The present invention further relates to the process for efficient production of levan by optimizing media parameters at a microlitre level.

### CROSS-REFERENCE TO RELATED APPLICATION

This Non-provisional application claims priority under 35 U.S.C. § 119(a) to India Patent Application No. 202211075840, filed on 27 Dec. 2022, the entire contents of which is hereby incorporated by reference in its entirety.

### FIELD OF THE INVENTION

The present invention relates to a process for the production of levan essentially free of contaminating polysaccharides using a micro-bioreactor. The present invention further relates to a process for the increased production of levan using a strain of marine Bacillus sp. SGD-03. Wherein, about 5-6 fold increase in the production of levan (20-30 g/L-120-130 g/L) was observed.

### BACKGROUND AND PRIOR ART OF THE INVENTION

Exopolysaccharides (EPS) are high-molecular weight polymers that are composed of sugar residues and are secreted by many microorganisms into the surrounding environment. Microorganisms synthesize a wide spectrum of multifunctional polysaccharides including intracellular polysaccharides, capsular polysaccharides and extracellular polysaccharides (EPS). Exopolysaccharides generally consist of (modified) monosaccharides and, tentatively, some non-carbohydrate substituents, such as acetate, pyruvate, succinate, and phosphate. One of the Exopolysaccharides includes levan polysaccharide.

Levan is a biopolymer of fructose linked by β-(2, 6) glycosidic bonds. It also has some β-(2, 1) linked branches. Levan differs from other polysaccharides in that it had been used as an emulsifier and thickener because it does not swell in water. Besides the common features of natural polysaccharides like safety, biocompatibility, and biodegradability, levan also exhibits many favorable properties such as moisturizing, anti-obesity, antitumor, antioxidant, anti-inflammatory, hyperglycemic inhibitor, and other biomedical functions. Therefore, levan has been widely applied in food, prebiotics, cosmetics, and medicines.

Nowadays, the industrial production of levan is carried out by different types of microorganisms. The microbial levans are produced from a sucrose-based substrate by transfructosylation reaction of a levansucrase (Beta-2,6-fructan:D-glucose 1-fructosyltransferase, EC 2.4.1.10). Bacillus subtilis is a known promising levan producer as it ferments sucrose with high levan production ratios. B. subtilis is also well-known for the production of commercially secondary metabolites like antibiotics and enzymes.

Chinese patent Application No 102168121 discloses a method for fermentative production of levan using Bacillus licheniformis.

U.S. Pat. No. 5,547,863 discloses a method for the increased production of levan essentially free of contaminating polysaccharides using Bacillus polymyxa. The method produces large quantities of a pure and uniform extracellular polysaccharide fructan (levan), in a sucrose medium.

Xu, Z., et al (2019), International journal of biological macromolecules, 141, 298-306. DOI: https://doi.org/10.1016/j.ijbiomac.2019.08.217 disclose a method for the isolation and structural characterization of exopolysaccharides produced from Bacillus licheniformis.

Several studies have been conducted for the production of levan using various microorganisms; however, most of them are challenged with the low yield of levan and contaminating of impure products. Regardless of many interesting characteristics and advantages showed by levan, the requirements of producing it on large scale increased dramatically.

Thus, there is still a need of process for efficient production of levan by optimizing media parameters at a microlitre level.

### OBJECTIVES OF THE INVENTION

The main objective of the invention is to provide a process for the production of levan essentially free of contaminating polysaccharides using a micro-bioreactor.

Another objective of the present invention is to provide process for synthesis of levan, wherein the levan described herein is a β-(2, 6) D-fructofuranosyl polymer produced by a strain of marine Bacillus sp. SGD-03.

Yet another object of the present invention is to provide process for synthesis of levan essentially free from other contaminating polysaccharide fermentation products at about three times the amount of previously known levan synthesizing microorganisms.

Still another objective of the present invention is to provide process for production of levan with enhanced yield by optimizing media parameters.

### SUMMARY OF THE INVENTION

This summary is provided to introduce a selection of concepts in a simplified form that are further described below in Detailed Description section. This summary is not intended to identify key features or essential features of the subject matter, nor is it intended to be used as an aid in determining the scope of the subject matter.

The present invention solves the challenges of the low yield and impurity of levan produced by the known processes. In a general aspect, the present invention provides a process for the production of levan which is essentially free of contaminating polysaccharides by using a high throughput micro bioreactor system.

Accordingly, the present invention provides a process for production of a levan essentially free of a contaminating polysaccharide using a micro-bioreactor, comprising the steps of:
a. preparing and optimizing a media comprising 4 to 10% sucrose using a microtiter plate in a micro-bioreactor;
b. inoculating a marine Bacillus sp. SGD-03 in the said media and incubating at 28ºC for 18-24 hours;
c. centrifuging the incubated media at 10000 rpm to obtain a cell-free supernatant;
d. precipitating the cell free supernatant using a solvent in absolute ethanol to obtain precipitated exopolysaccharide (EPS);
e. separating the precipitated EPS in absolute ethanol by centrifugation followed by washing with a solvent;
f. dissolving washed EPS of step e) in a solvent to make a solution containing levan; and
g. lyophilizing the solution containing levan.

In an embodiment of the present invention the yield of the levan by the above process is in range of 107 to 123 g/lt with 99% of purity.

In an embodiment of the present invention the molecular weight of the levan is in the range of 1.0×10⁴ kDa.

In an embodiment of the present invention the media is a composition of polysaccharides, peptone, beef extract and salt.

In a preferred embodiment of the present invention the media comprises of 0.4% of peptone, 0.4% of beef extract, 0.5% of NaCl, 4% of sucrose and water.

In an embodiment of the present invention the marine Bacillus sp. SGD-03 is grown for 8-14 hrs in a rotatory shaker at 150 rpm for 12-18 h to prepare the inoculum.

In an embodiment of the present invention the solvent for precipitation is three-volume of pre-chilled absolute ethanol (4° C.).

In an embodiment of the present invention the EPS is dissolved using solvent Milli Q H₂O for lyophilization.

In an embodiment of the present invention the diameter of levan fibers

obtained is in the range of 3.620 μm to 9.071 μm.

In an aspect of the present invention, the inoculation of marine Bacillus strain culture in said media is 1%.

In an aspect of the present invention, the precipitating step may be done in any size of Eppendorf tube as per requirement, specifically done in 2 mL of Eppendorf tube.

### BRIEF DESCRIPTION OF THE DRAWINGS

The invention has other advantages and features which will be more readily apparent from the following detailed description of the invention, when taken in conjunction with the accompanying drawings, in which:

FIG. 1: depicts the main effects of variable on EPS production, in accordance with an embodiment of the present disclosure.

FIG. 2: FIG. 2A, FIG. 2B, FIG. 2C, and FIG. 2D depict the Response of variables and 3D scatter plots on EPS production of: A) Peptone and beef extract Vs EPS, B) Sucrose, peptone Vs EPS, C) Sucrose; beef extract Vs EPS, and D) 3D scatter plot of central composite design data points, in accordance with an embodiment of the present disclosure.

FIG. 3: FIG. 3A, FIG. 3B, FIG. 3C depict the microbiolector comparative validation for EPS production: BioLector (1.0 ml) Vs Fermentor (1.0-10.0 L), in accordance with an embodiment of the present disclosure.

FIG. 4: depicts the effect of different carbon sources on EPS production, in accordance with an embodiment of the present disclosure.

FIG. 5: depicts the effect of sucrose concentration on EPS production, in accordance with an embodiment of the present disclosure.

FIG. 6: depicts the correlation of EPS and Biomass, in accordance with an embodiment of the present disclosure.

FIG. 7: FIG. 7A and FIG. 7B depict the Scanning Electron Micrograph of: A) Standard EPS from Ervinia herbicola, B) Test EPS from Bacillus sp. SGD-03, in accordance with an embodiment of the present disclosure.

FIG. 8: depicts the TLC chromatogram of Fructose and hydrolyzed EPS, in accordance with an embodiment of the present disclosure.

FIG. 9: depicts the HPLC chromatogram, in accordance with an embodiment of the present disclosure.

FIG. 10: depicts the functional group determination by FT-IR spectrogram of the EPS produced by marine strain, in accordance with an embodiment of the present disclosure.

FIG. 11: depicts the size exclusion chromatogram of the Bacillus sp. SGD-03 EPS, in accordance with an embodiment of the present disclosure.

FIG. 12: depicts the linear graph of standard pullulan polysaccharides, in accordance with an embodiment of the present disclosure.

FIG. 13: depicts Column purified EPS, in accordance with an embodiment of the present disclosure.

### DESCRIPTION OF THE INVENTION

The invention has other advantages and features which will be more readily apparent from the following detailed description of the invention, when taken in conjunction with the accompanying drawings, in which:

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The tables, figures and protocols have been represented where appropriate by conventional representations in the drawings, showing only those specific details that are pertinent to understanding the embodiments of the present invention so as not to obscure the disclosure with details that will be readily apparent to those of ordinary skill in the art having benefit of the description herein.

Accordingly, to accomplish the objectives of the invention, the present invention discloses a process to increase the production of levan by using a high throughput micro bioreactor system, wherein, the Levan is essentially free of contaminating polysaccharides.

In an embodiment, the levan which is essentially free of contaminating polysaccharides is produced by using a high throughput micro bioreactor system and strain of marine Bacillus sp. SGD-03 i.e., β-(2, 6) D-fructofuranosyl polymer.

In an embodiment of the present invention, the levan produced from marine Bacillus sp. SGD-03 also has cholesterol-lowering properties and it can also be employed as prebiotic and probiotic either alone or in combination with other agents.

In still another embodiment of the present invention, the process of preparation of levan using Bacillus sp. SGD-03 strain is a cost-effective robust process.

In an embodiment of the present invention, the levan described herein is a β-(2. 6) D-fructofuranosyl polymer produced by using a strain of marine Bacillus sp. SGD-03.16s rRNA gene sequences of strain Bacillus sp. SGD-03 has been deposited in NCBI Gene Bank having the accession no. KF265353. The Bacillus sp. SGD-03 has been deposited at NCMR (National Centre for Microbial Resource), National Centre for Cell Science (NCCS), Pashan, Pune, Maharashtra-411021, INDIA having the accession no. MCC 0243 on 9 Nov. 2021.

In another embodiment of the present invention, an enhancement in exopolysaccharide yield is obtained by optimizing the growth media parameters using various factors at a micro liter level by using microbioreactor. The optimized media parameters are studied and sucrose is one of the factors found to be the best carbon source having a significant effect on EPS production in Bacillus sp. SGD-03.

In still another embodiment of the present invention, sucrose is required in growth media for levan synthesis and is optimized at a concentration between 4-10%.

In yet another embodiment of the present invention, the sucrose in growth median is undiluted and acts as a substrate for the EPS production and it is required for the growth of Bacillus sp. SGD-03.

In still another embodiment of the present invention, the growth medium comprising factor beef extract and factor peptone showed maximizing effect on EPS production.

It has been found that the factor sucrose with high percentage 4.0 along with 0.4 percent of the individual factor beef extract and peptone showed maximizing effect on EPS production.

In an embodiment of the present invention, yield of EPS produced from Bacillus sp. SGD-03 in presence of sucrose is almost increased by 10-fold as compared to other sources such as glucose, fructose, lactose and maltose. In another embodiment of the present invention, the Bacillus sp. SGD-03 strain produces 120-130 g/L of EPS, which is approximately 1.12 USD/L of media cost.

In yet another embodiment of the present invention, the produced EPS from the Bacillus sp. SGD-03 strain has been identified as levan with a molecular weight of 1.0×10⁴ Da.

In still another embodiment of the present invention, the Bacillus sp. SGD-03 strain produces a levan in a yield of 107-123 g/L.

In still another embodiment of the present invention, the synthesized levan has formed fiber diameters in the range of 3.620 μm to 9.071 μm.

In still another embodiment of the present invention, the process synthesizes Levan-EPS within 24 hr.

In an embodiment of the present invention, the levan produced from the marine Bacillus sp. SGD-03 also has cholesterol-lowering properties (Table 3).

In still embodiment of the present invention, the process of preparation of levan using Bacillus sp. SGD-03 strain is a cost-effective robust process.

In an embodiment of the present invention, enhanced production of exopolysaccharide is obtained by optimizing media parameters at a micro liter level by using microbioreactor. The optimized process parameters are found by following steps:
a) Isolation and Identification of a Levan Producing Microorganism;
b) Screening and Identification of EPS positive strains;
c) Optimization of optimum process parameters for EPS production using BioLector Pro screening of carbon source for EPS production;
d) Response surface methodology (RSM).

The process for production of Levan using Bacillus sp. SGD-03 strain with optimized process parameters comprises the steps of:
a) optimizing a media using optode embodied 48-well microtiter plate;
b) growing cultures overnight (12-18 hrs) in said media in a rotary shaker at 150 rpm for 12 to 18 hours;
c) inoculating 1% of said Bacillus sp. SGD-03 strain in said culture;
d) incubating the culture of step c) at 28° for 18 to 24 hours followed by centrifugation at 10000 to obtain cell-free supernatant;
e) precipitating the obtained supernatant of step d) using solvent absolute ethanol;
f) separating the solid precipitated matter of step f) followed by washing with a solvent;
g) dissolving the washed solids of step g) in a solvent;
h) quantifying levan (EPS) of step h) by carbohydrate quantification assay (phenol sulphuric acid assay); and
i) lyophilizing and storing the as synthesized levan. wherein, step a) media optimization was performed using optode embodied 48-well microtiter plate (Flower well plates, m2p-labs GmbH, Germany) in a BioLector machine, which facilitates online monitoring of cell biomass, pH, and dissolved oxygen (DO) saturation separately in each well without effusing (Kensy, F., Zang, et al. (2009), Validation of a high-throughput fermentation system based on online monitoring of biomass and fluorescence in continuously shaken microtiter plates. Microbial cell factories, 8(1), 1-17, doi: 10.1186/1475-2859-8-31),; Dos Santos, et al. (2016), An improvement of surfactin production by B. subtilis BBG131 using design of experiments in microbioreactors and continuous process in bubbleless membrane bioreactor. Bioresource technology, 218, 944-952. DOI: https://doi.org/10.1016/j.biortech.2016.07.053) and wherein, step b) cultures are overnight grown in rotatory shaker at 150 rpm for 12-18 h.

The step of inoculation is carried out, wherein 1.0% inoculum was added in said media.
Wherein, the incubation is carried out at 28° C. for 18 to 24 h.
Wherein, the step of centrifugation is carried out at 10000 rpm, for 20 to 30 min at room temperature to obtain cell-free supernatant.
Wherein, the step of precipitation is carried out by using three-volume of pre-chilled absolute ethanol (4° C.), which is added to the supernatant and which result in the formation of precipitate which was then separated and washed with 70% ethanol.

In an aspect of the present invention the step of precipitation can also be carried out using solvent selected from isopropanol, acetone, or methanol.

In specific embodiment, the media used essentially comprises of 40 g/L of sucrose, 4 g/L of beef extract, and 4 g/L of peptone, without any minerals, which is a cost-effective option.

In specific embodiment, the sucrose is used as the sole carbon source in the media for levan production.

In another specific embodiment, the process produces a levan with uniform molecular weight of 10 kDa, without any variation in the molecular weight, and which can be used for specific applications. Moreover, 4% of sucrose as carbon source, yields 123.9 g/L of partially/fully purified levan, confirming a high substrate ratio to the product formation.

In another specific embodiment, the process produces levan with 107-123 g/L, without any contaminating polysaccharides, by using lesser sucrose i.e., 40 g/L concentration, by using an organism isolated from a marine environment, wherein the hydrolysis is done for 1 hrs.

Generally, the production of levan in shake flask and batch fermentation methods requires continuous attention till the completion of the process. The shake flask studies require human resource, a high amount of media and separate utilities to optimize each process parameter and set of experiments. A micro bioreactor as covered in present invention offers a high throughput optimization of process parameters in one go, which helps see the interaction of all process parameters simultaneously. Sometimes there is a variation of production yield of levan with different days of the experiment, which may be caused due to the intervention of atmospheric conditions (temperature, humidity, and moisture). To overcome the problem, a high throughput automatic operational device that can run multiple sets of experiments in one go minimizes the variation of experimentation obtained from different days and be the best alternative.

In the present invention, the experiment design can generate all possible combinations of experiments. It can overcome the problem associated with production with one factor at a time (OFAT). In addition, a multimode microplate reader efficiently analyses 96 samples at a time, which adds an advantage to the process. Employing such techniques in sequential order could produce the product with minimum requirements. Utilizing the design of an experiment tool to perform experiments in high throughput, an automated device that minimizes media cost and time is a prerequisite of today's era. In addition to this, with the help of experiment design and online monitoring of the process can be achieved using a micro bioreactor.

Although the subject matter has been described in considerable detail with reference to certain examples and implementations thereof, other implementations are possible. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The following examples, which include preferred embodiments, will serve to illustrate the practice of this invention, it is being understood that the particulars shown are by way of example and for purpose of illustrative discussion of preferred embodiments of the invention.

The sucrose, peptone, beef extract, and sodium chloride as disclosed in the present invention are obtained from Hi-media, Mumbai.

### Example 1

### Isolation and Identification of a Levan Producing Microorganism

Microbial cultures were isolated from marine sediment samples from Chorao Island, Goa, and were used for EPS screening.

### Screening and Identification of EPS Positive Strains:

A total of 43 marine strains were screened for the EPS production. Four strains were found positive based on screening. Out of 4 positive strains for EPS production SGD-03 was found to be the best EPS producer. Bacillus sp. SGD-03 was found to produce mucous slimy colonies on the solid medium. Colonies with the presence of exopolysaccharide were found precipitating upon addition of pre-chilled absolute ethanol, a second confirmatory parameter for EPS production. Upon lifting the colonies with a sterile inoculating loop an approximately 7-8 mm string was formed which further confirms the presence of EPS. According to Hector et al. 2015, String of >5 mm colonies can be considered positive for EPS production (Hector, S., et al, 2015. Diverse exopolysaccharide producing bacteria isolated from milled sugarcane: implications for cane spoilage and sucrose yield. PLOS One, 10(12), e0145487. doi:10.1371/journal.pone.0145487). Microscopic observation has shown that SGD-03 belongs to the gram-positive, rod-shaped bacterium. Based on 16S rRNA sequencing and NCBI-BLAST and EzBioCloud database analysis, a phylogenetic tree was constructed to measure evolutionary distance of SGD-03 with type strains, the SGD-03 has shown the highest sequence similarity of 99.79% with Bacillus licheniformis ATCC 14580. An ANI matrix value, 99.0% has also shown that the genome relatedness with type strains as well as ANI plot showed its similarity with type strains. Thus, the strain was identified as Bacillus licheniformis and named Bacillus sp. SGD-03. The tolerance of salt and pH was analyzed on basis of the growth profile of an organism. It was observed that the Bacillus sp. SGD-03 is growing well in salt conditions. An inverse relationship of growth to salt concentration was observed. As the concentration of salt increases, the growth of an organism decreases. Optimum growth of an organism was found at 2.0% concentration of salt while, on the observation of pH, it was found that the growth of an organism was greatly affected at low pH 4.0 and high pH 10.0. The pH 6.0 and 7.0 is the optimum range for the growth of an organism. A moderate growth profile was observed at pH 5.0, 8.0, and 9.0. It can be concluded that the organism can tolerate a wide range of pH as well as salt.

### Optimization of Optimum Process Parameters for EPS Production Using BioLector Pro Screening of Carbon Source for EPS Production:

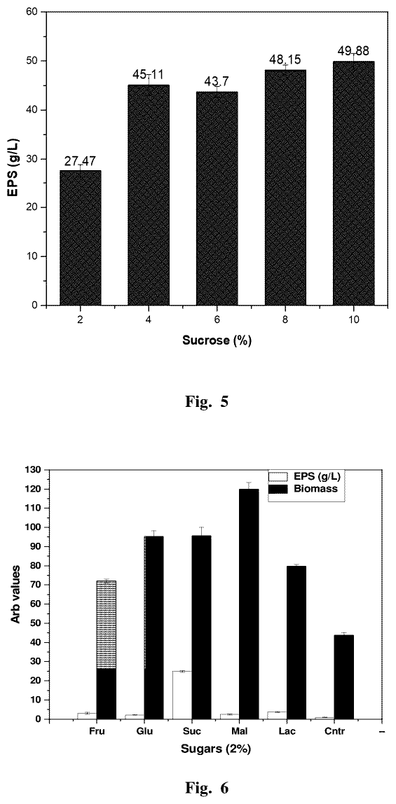

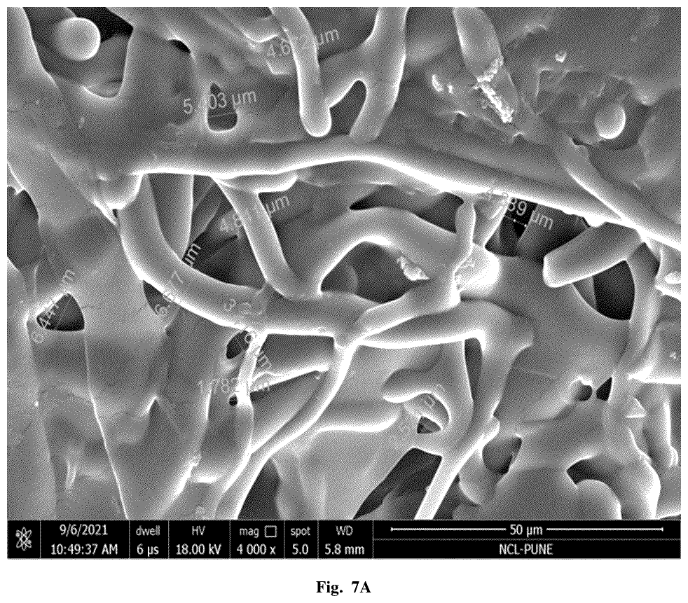

The influence of various carbon sources on the EPS production was investigated using a micro bioreactor i.e. BioLector Pro. Five different carbon sources including monosaccharide (fructose, glucose) and disaccharide (sucrose, maltose, and lactose) were studied to examine their effect on EPS production. Sucrose was found to be the best carbon source having a significant effect on EPS production. Sucrose showed almost a 10-fold increase in EPS production as compared to other sources (FIG. 4). The 4.0% sucrose concentration was found to be an optimum concentration on EPS production. Though the observation seems direct relationship of EPS production with increasing concentration of sucrose, there is no significant increase in the EPS yield with the higher percentage of sucrose (FIG. 5). The correlation between biomass in respect to different carbon sources with EPS production using BioLector Pro was showed in FIG. 6. Glucose and sucrose were demonstrated a similar pattern on the growth profile of an organism but varies for EPS production while maltose has shown a significantly great growth profile but not the EPS production (FIG. 6).

### Screening of Significant Variables:

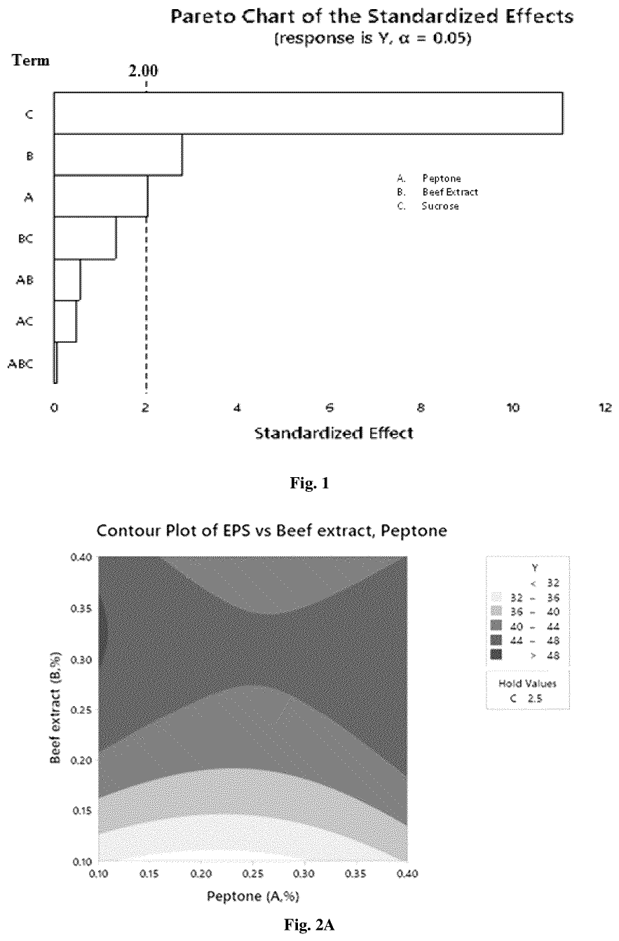

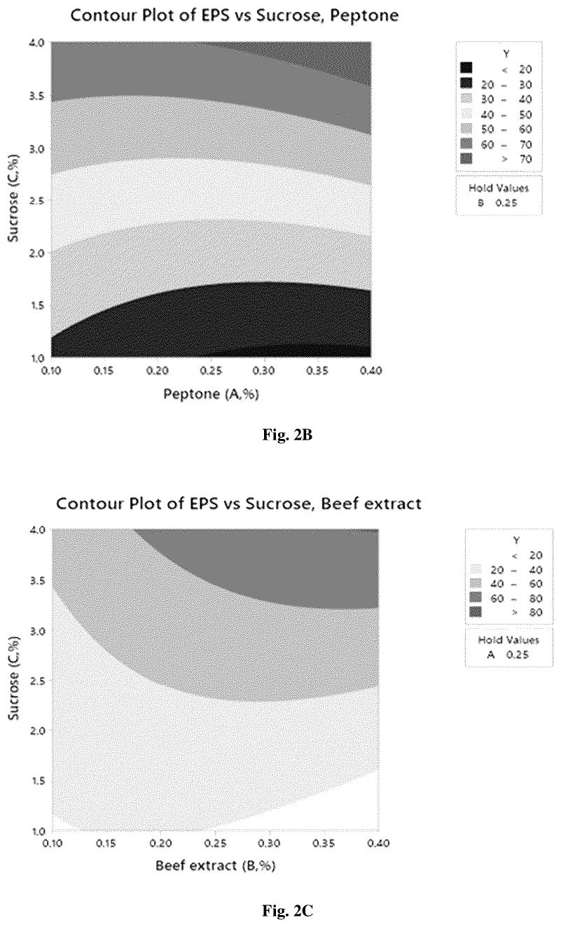

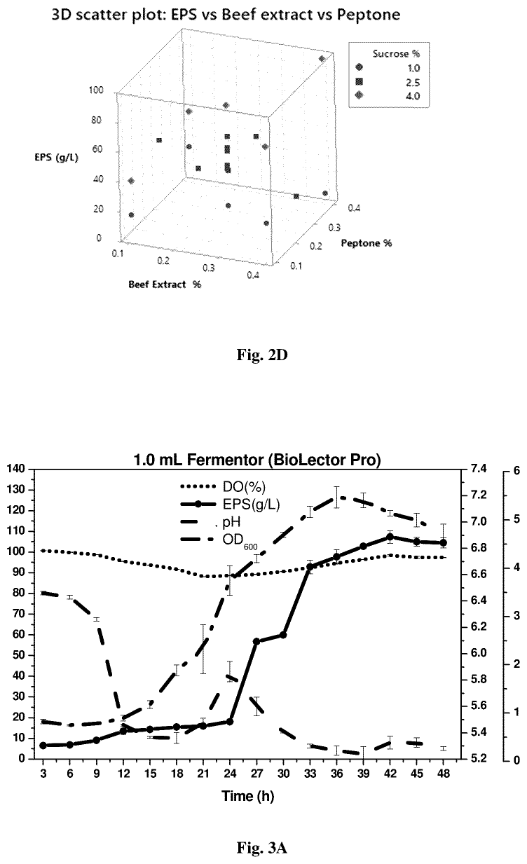

To choose the best combination of nutrient broth components i.e., peptone, beef extract, and NaCl with an effect of supplemented carbon source, sucrose was screened from low to high 0-3.0% concentration. The significant variables were obtained by the full factorial design with regards to influence on EPS production. The results were interpreted by linear regression analysis using the Minitab 19 statistical software (Crack version) which helps to estimate the effect of each independent variable. The sucrose with a higher concentration has the main effect on EPS production (FIG. 1). Peptone and beef extract were also found to have a positive impact to enhance EPS production. (FIG. 2A).

Statistical significance of the effect of each variable on EPS production was calculated and investigated by F-test which has shown adequacy of the design with highlighting all the factors i.e., factor (A), Peptone; factor (B), beef extract, and factor (C), sucrose are having the statistically significant effect on EPS production. Based on this observation it can be concluded that all the factors have their importance in the production of EPS. The p-value <0.000 confirms that the overall model is very significant and productive. Moreover, the value of R2 0.584 for the model indicates that the 40% variation in the sample indicates the results of individual variables effect. Therefore total variance could not be explained by the model. The probable cause could be the various interactions of variables as mentioned in Table 1 (Dos Santos et al (2016), An improvement of surfactin production by B. subtilis BBG131 using design of experiments in microbioreactors and continuous process in bubbleless membrane bioreactor. Bioresource technology, 218, 944-952 DOI: https://doi.org/10.1016/j.biortech.2016.07.053). The response of each run was calculated using the following regression equation:

$$Y = 2.13 + 2.83 A + 2.27 B + 8.31 C - 0.84 A * B - 0.15 A * C + 1.29 B * C + 0.236 A * B * C$$

Where, Y=Response, A=Peptone, B=Beef extract and C=Sucrose

TABLE 1
Analysis of variance for anticipation of EPS-Biomass yields
influencing by variables in screening design of experiment.

| Source | DF | Adj SS | Adj MS | F-Value | P-Value |
|---|---|---|---|---|---|
| Model | 7 | 20965.0 | 2995.0 | 24.12 | 0.000 |
| Linear | 3 | 20352.5 | 6784.2 | 54.63 | 0.000 |
| A | 1 | 566.9 | 566.9 | 4.57 | 0.035 |
| B | 1 | 1936.8 | 1936.8 | 15.60 | 0.000 |
| C | 1 | 17848.8 | 17848.8 | 143.74 | 0.000 |
| 2-Way Interactions | 3 | 598.6 | 199.5 | 1.61 | 0.191 |
| A × B | 1 | 47.3 | 47.3 | 0.38 | 0.538 |
| A × C | 1 | 8.6 | 8.6 | 0.07 | 0.793 |
| B × C | 1 | 542.6 | 542.6 | 4.37 | 0.039 |
| 3-Way Interactions | 1 | 13.9 | 13.9 | 0.11 | 0.739 |
| A × B × C | 1 | 13.9 | 13.9 | 0.11 | 0.739 |
| Error | 120 | 14901.2 | 124.2 | | |
| Lack-of-Fit | 56 | 12654.6 | 226.0 | 6.44 | 0.000 |
| Pure Error | 64 | 2246.6 | 35.1 | | |
| Total | 127 | 35866.2 | | | |

Microorganisms produce EPS in response to various stress conditions like nutrient deficiency, salt tolerance, heat tolerance, drought conditions, etc (Sandhya, V., & Ali, S. Z. (2015). The production of exopolysaccharide by Pseudomonas putida GAP-P45 under various abiotic stress conditions and its role in soil aggregation. Microbiology, 84, 512-519, 10.1134/S0026261715040153). According to this, the percentage of selected optimum levels of input factors was modified by diluting 10 times to mimic the nutrient deficient and salt tolerance conditions to induce the organism's EPS producing ability to maximize the yield of EPS. The concentration of sucrose was used undiluted as the carbon source acts as a substrate for the EPS production and is required for the growth of an organism.

### Response Surface Methodology (RSM):

The selected optimum levels of a factor with some modifications were further optimized using face-centered composite design (FCCD). A total number of 20 different run with various combinations of peptone, beef extract, and sucrose in a set of factorials, axial and center points. The response of each run was calculated using the following regression equation:

$$Y = 29.1 - 117 A + 73 B - 5.7 C + 161 A * A - 313 B * B + 0.78 C * C - 72 A * B + 22.6 A * C + 53. B * C$$

Where, Y is the response variable; A, peptone; B, Beef extract & C is the sucrose.

The results of variance (ANOVA) were analyzed to check the significance of the fit of the first-order polynomial equation of the FCCD experimental data. The model coefficient of determination R² value was calculated as 0.831 which indicates that the 83.11% variability in the response and better explained by the model. The model's fisher F values of 5.47 and the p values <0.007 indicate the model is statistically significant (Table 2). The optimum level of each variable and the effect of their interaction on response were studied by the 2D contour plots counter to any two independent variables withholding other variables at a constant of and 3D scattered plot. The interaction of beef extract and peptone showed that they are equally important to maximize EPS production (FIG. 2A). The factor sucrose with the high percentage, 4.0 along with 0.4 percent of the individual factor beef extract and peptone showed the maximizing effect on EPS production. This combination of media found best which can produce 97.6 g/L of EPS (FIG. 2B, 2C, 2D).

TABLE 2
Analysis of variance of & their influence
on response by CCD experimental design

| Source | DF | Adj SS | Adj MS | F-Value | P-Value |
|---|---|---|---|---|---|
| Model | 9 | 8404.8 | 933.87 | 5.47 | 0.007 |
| Linear | 3 | 6897.1 | 2299.02 | 13.46 | 0.001 |
| A | 1 | 0.6 | 0.64 | 0.00 | 0.952 |
| B | 1 | 303.0 | 303.04 | 1.77 | 0.212 |
| C | 1 | 6593.4 | 6593.39 | 38.59 | 0.000 |
| Square | 3 | 139.9 | 46.62 | 0.27 | 0.844 |
| A × A | 1 | 36.1 | 36.10 | 0.21 | 0.656 |
| B × B | 1 | 136.3 | 136.34 | 0.80 | 0.393 |
| C × C | 1 | 8.5 | 8.46 | 0.05 | 0.828 |
| 2-Way Interaction | 3 | 1367.9 | 455.95 | 2.67 | 0.105 |
| A × B | 1 | 21.3 | 21.28 | 0.12 | 0.731 |
| A × C | 1 | 207.6 | 207.59 | 1.21 | 0.296 |
| B × C | 1 | 1139.0 | 1138.98 | 6.67 | 0.027 |
| Error | 10 | 1708.6 | 170.86 | | |
| Lack-of-Fit | 5 | 1263.7 | 252.75 | 2.84 | 0.138 |
| Pure Error | 5 | 444.9 | 88.98 | | |
| Total | 19 | 10113.4 | | | |

### Validation of the Model at 1 L and 10 L Fermenters Level

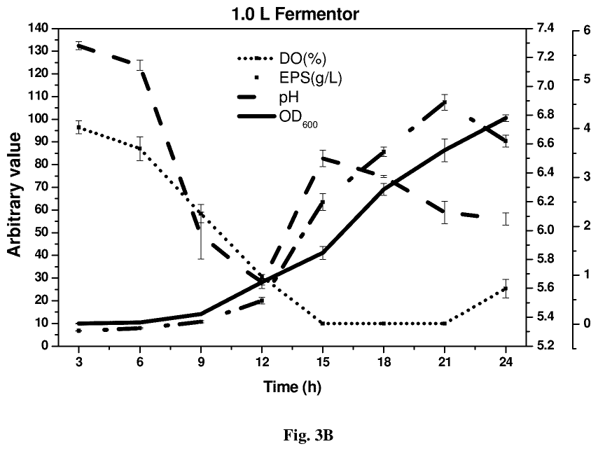

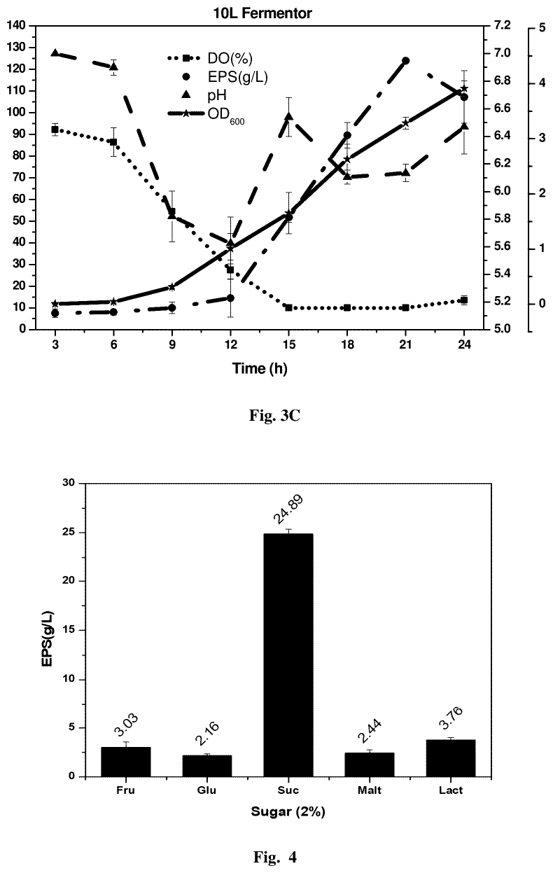

To validate the continual of the model for predicting conditions and optimum response values, the model was validated with the selected optimal conditions of the full factorial and central composite design in 500 mL of Erlenmeyer flask containing 200 mL of the optimal composition of media followed by 1.0 L and 10.0 L fermenters. Under the suggested conditions, the response value of EPS yield was 97.6 g/L. The results of FFD and CCD with respect to BioLector have shown the variation of ±1.6 in shake flask studies and ±4.79 g/L, respectively (data not shown), which confirms the variation in the production and the reproducibility of the model process parameters. The comparative result of fermenters has also confirmed the significance of the model. The value of microbioreactor (BioLector Pro), 1.0 L and 10.0 L fermenter level had shown the variation of ±9.9 g/L and ±23.6 g/L of EPS. The variation of ±9.9 g/L and ±23.6 g/L of EPS in microbioreactor to fermentor may be the cause of continuous supply and maintaining 10% oxygen saturation. In the validation at large scale (1.0 L and 10.0 L) fermentation, optimum EPS synthesis was observed at 21 h and start decreasing after 21 h. Increasing dissolve oxygen saturation after 21 h indicates that organisms are dying due to nutrient depletion and at the same time the optical density of organisms appeared Increasing. It could be concluded that organisms maintaining a stationary phase by utilizing EPS as a carbon source and it is expected that once the carbon source depletes, the organism starts utilizing EPS as a carbon source for its survival. The common condition in fermentation, as the organism, metabolizes the nutrients, various organic acids are produced which is indicating by a drop in pH, while pH increases as a result of ammonia production initiate upon utilization of proteins (FIG. 3).

### Example 2

### Synthesis of Levan

Synthesis of levan and Media optimization was performed using optode embodied 48-well microtiter plate (Flower well plates, m2p-labs GmbH, Germany) in a BioLector machine, which facilitates online monitoring of cell biomass, pH, and dissolved oxygen (DO) saturation separately in each well without effusing (Kensy, F., et al (2009). Validation of a high-throughput fermentation system based on online monitoring of biomass and fluorescence in continuously shaken microtiter plates. Microbial cell factories, 8(1), 1-17. doi:10.1186/1475-2859-8-31; Dos Santos, et al. (2016), An improvement of surfactin production by B. subtilis BBG131 using design of experiments in microbioreactors and continuous process in bubbleless membrane bioreactor, Bioresource technology, 218, 944-952, https://doi.org/10.1016/j.biortech.2016.07.053). With working volume 1.0 mL, overnight grown culture (18 h), inoculated as 1% (0.1 OD600) in each well containing various media combinations generated by experimental design. (Media components peptone, beef extract, and sucrose were prepared in stock concentrations, pH was adjusted to 7.0). The plate was fixed and ran at a temperature of 28° C.; rpm, 1400; humidity, 100% for 48 h of incubation. Hereafter, the plate was taken out from the microbioreactor. Samples were centrifuged at 10000 rpm, 30 min at room temperature to obtained cell-free supernatant. The three-volume of pre-chilled absolute ethanol (4° C.), was added to the supernatant Precipitated EPS (Threaded appearance) was separated and washed with 70% ethanol. Then 1 mL of Milli Q water was added and kept overnight at 4° C. for proper dissolution. For the process validation, the optimized media composition of Full Factorail Design (FFD) and Central Composite Design (CCD) for EPS production were subjected to a shake flask (500 mL) followed by 1.0-10.0 L fermenters. Erlenmeyer flask of 500 mL containing 200 mL of working volume was set. The flasks were inoculated and incubated at 28° C. at 150 rpm for 24 h. Dissolved oxygen saturation, 10% was maintained by cascading rpm from 250 to 500; pH 7.0; temperature, 28° C., and 24 h of incubation were set for fermenter scale (1.0 L and 10.0 L). Steps from harvesting to dissolution were repeated. EPS was diluted and quantified by the phenol sulphuric acid method.

### Example 3

### Characterization of Levan

### Field Emission Scanning Electron Microscopy (FE-SEM) Analysis

The surface morphology of levan was observed and analyzed using FE-SEM. Dry polysaccharide samples (Test and standard) were mounted over a carbon tape fixed on the sample holder stub. Prior to analysis, samples were subjected to gold coating using a sputter coater. The surface morphology was observed and photographed from 1000× to 4000× magnification with an accelerating voltage of 18 kV using the FEI Nova NanoSEM 4450 electron microscope.

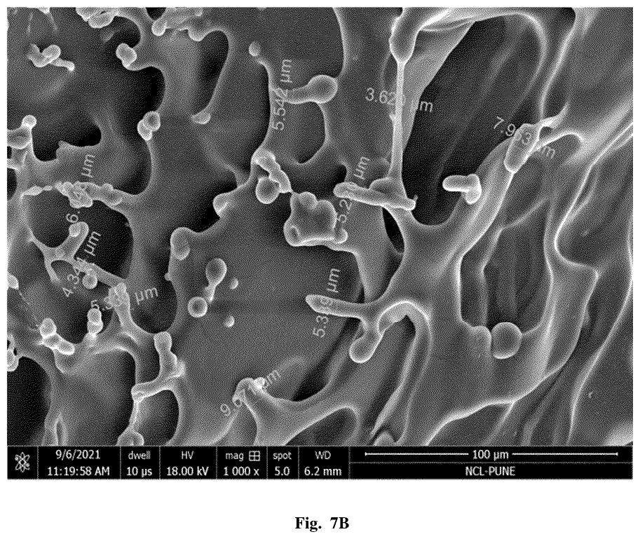

The scanning electron micrographs showed a significantly fibrous network structure with a relatively smooth surface of polysaccharides HPLC and TLC have revealed that the EPS belongs to fructan class polysaccharide. Accordingly, the Surface morphology of test EPS was compared with standard EPS (Levan) of Erwinia herbicola. A smooth surface was observed in the standard EPS (FIG. 7a) as well as test EPS (FIG. 7b) with various ranges of fiber diameters. The fiber diameters were found in the range of 3.620 μm to 9.071 μm forming various pore sizes of the structure. Test EPS showing quite different morphology and fibrillar diameter than standard. Variation in fiber diameter may due to the various degrees of polymerizations from organism to organism.

### Monosaccharide Composition Analysis

Monosaccharide analysis of EPS (levan) was performed by thin-layer chromatography (TLC), high-performance liquid chromatography (UHPLC), and liquid chromatography high-resolution mass spectrometry (LC-HRMS). EPS of 1.0 mg/mL was hydrolyzed using 2N trifluoroacetic acid (TFA) at 100° C. for 1 h with intermediate shaking. Hydrolysates were neutralized by evaporating TFA using vacuum drying. Traces of TFA were removed by adding methanol followed by water until it neutralizes. The hydrolyzed sample was centrifuged at the highest rpm to remove unhydrolyzed or charred material, the supernatant was taken out carefully and subjected to analyze the carbohydrates.

### Thin Layer Chromatography

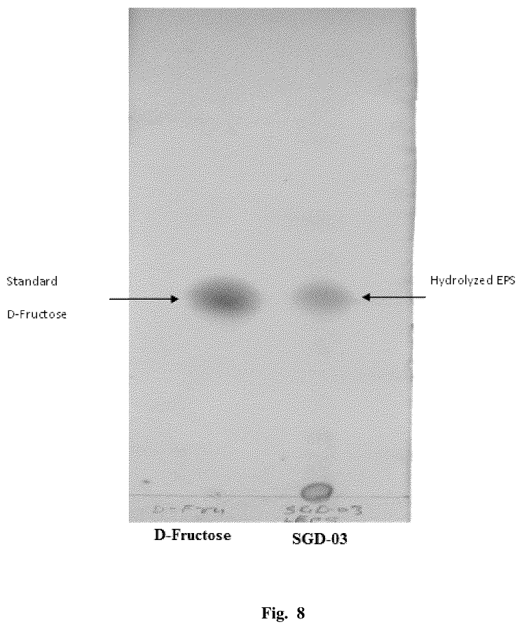

Fructose was used as a standard carbohydrate Standard carbohydrate along with hydrolyzed sample, 5.0 μL was spotted on TLC plate as stationary material (Silica 60, Merck), air-dried, and placed in a TLC chamber saturated with the mobile phase. Solvent system acetonitrile: water in the ratio of 85:15 (v/v) was used to separate the carbohydrates. The plate was removed, air-dried, and subjected to develop chromatogram in the staining solvent p-anisaldehyde: sulphuric acid: ethanol with 1:1:20 ratio. Then the chromatogram was visualized by charring the plate in a hot air oven for 10 min at 110° C. (FIG. 8).

### High-Performance Liquid Chromatography

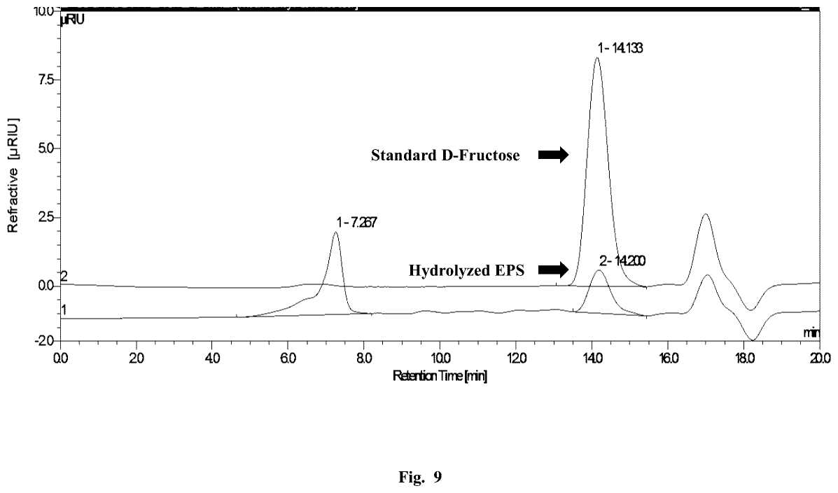

The hydrolyzed sample was filtered using a 0.22 μm syringe filter and subjected to Ultra high-performance liquid chromatography (UHPLC, Ultimate 3000, ThermoFisher) equipped with Phenomenex (Rezex RCM-Monosaccharide) column set on 80° C. Millipore ultrapure H2O as a carrier with a flow rate of 0.6 mL/min, RI at 35° C. conditions were used for detection. An injection volume of 20 μL having a concentration of 1000 ppm was injected along with standard carbohydrates for comparison (FIG. 9).

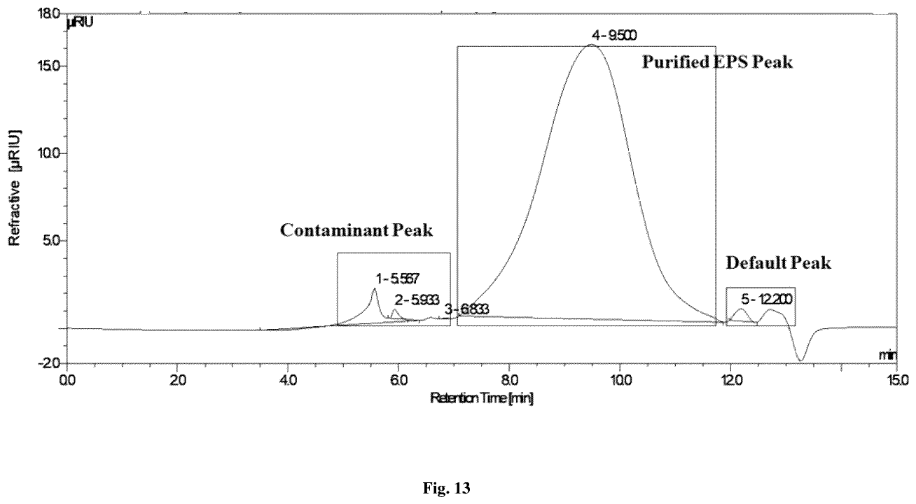

The extra pure levan was fractionated using HPLC, equipped with a size exclusion column, diol 200 (YMC). Column-based purification of polysaccharide is the extended level of purification step. The fractionated levan can be directly subject to structural characterization. The real-time monitoring of HPLC chromatogram and fractionation confirms the absolute purity of levan, which is free of other contaminants (refer FIG. 13).

### Fourier Transform Infrared (FTIR) Spectroscopy

Analysis of functional groups was carried out using FTIR spectroscopy KBr was activated in a hot air oven at 80° C. for overnight. The sample was mixed in KBr in a ratio of 1:100 (mg), ground to prepare the test KBr disc (13 mm). The disc was kept in desiccators to resist moisture entrapment and subjected to record absorption spectra. Spectra were recorded from the wavenumber 4000 cm⁻¹ to 400 cm⁻¹ using FT-IR spectrometer vertex70, Bruker. Background spectra were subtracted by recording spectra of reference KBr disc.

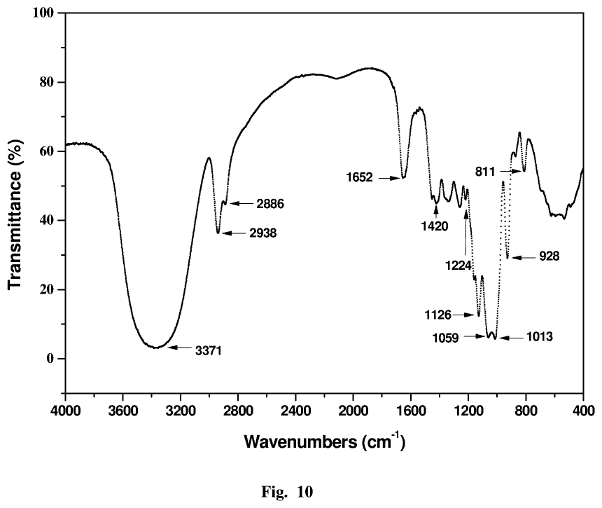

Various functional groups present in the polysaccharide were analyzed and assigned using the spectra of Fourier transforms infrared spectroscopy. Characteristic absorption spectra of polysaccharides were showed in the results (FIG. 10). The broad and strong intensity peak at 3371 cm⁻¹ corresponds to Hydroxyl (O—H) stretching, characterizing the polyhydroxy nature of polysaccharides. The weak absorption at 2938 cm⁻¹ and 2886 cm⁻¹ are corresponding peaks to C—H stretching, suggesting the presence of methylene groups vibrating asymmetric and symmetrically Further, an intense absorption at 1652 cm⁻¹ is the characteristic peak of carbonyl (C═O) stretching vibration it could be due to the aldehydic/ketonic nature of saccharides. Medium absorption peaks between 1420 cm⁻¹ to 1191 cm⁻¹ belong to deformation vibration of the C—H and O—H groups (Ibrahim et al., 2006). A medium intense peak of C—O—H vibration appears at 1126 cm⁻¹. An absorption peak at 1059 cm⁻¹ suggesting the presence of D-fructose saccharide. The strong absorption at 1013 cm⁻¹, C—O—C stretching is the indicative marker peak of the polysaccharides. The absorbance peak between 1000 to 800 cm⁻¹ belonged to typical carbohydrates. The peaks at 928 and 811 cm⁻¹ are corresponding peaks of the furanoid ring structure of the saccharide unit (FIG. 10).

### Example 4

### Molecular Weight Determination

Levan's average molecular weight was determined by the size exclusion chromatographic (SEC) column, combined with a UHPLC system with a RI detector. EPS sample 1.0 mg/mL was prepared in the mobile phase, Milli Q water, and filtered through a 0.45 μm syringe filter. An injection volume of 20 μL was passed through the column (Diol-200 YMC) with a flow rate of 1.0 mL/min at an ambient temperature. Standard Pullulan with an Mp ranging from ˜350 to 700,000 Dalton (Sigma) was used for calibration. The calibration curve was used to determine the average molecular weight of Levan, Test as well as sample chromatogram, was also compared with standard polysaccharides, Pullulan 10 kDa (P-10) and Dextran 10 kDa (D-10).

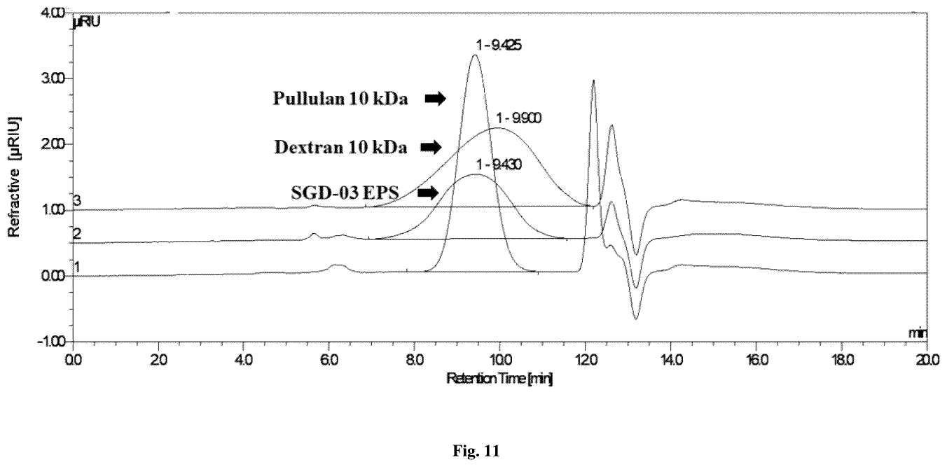

The average molecular weight of purified EPS was determined using size exclusion chromatography. The calibration curve for molecular weight determination was made using several standards of Pullulan polysaccharide ranging from ˜350-700 Mp. On the basis of calibration and comparing with standard Pullulan and Dextran, the size exclusion chromatogram has shown that the exopolysaccharide is made up of an average molecular weight of 1.0×10⁴ Da. Supplementing the initial concentration of sucrose, 40%, and consumption of about a quarter of sucrose, The Levan with less than 1.0×10⁴ Da molecular weight was produced (Tanaka, T., Oi, S., & Yamamoto, T. (1980). The molecular structure of low and high molecular weight levans synthesized by levansucrase, The Journal of Biochemistry, 87(1), 297-303. https://doi.org/10.1093/oxfordjournals.jbchem.a132737), so it can be assumed that upon utilization of 100% sucrose, the Levan with molecular weight, 1.0×10⁴ Da could be produced (FIG. 11).

### Example 5

### NMR Spectroscopic Analysis

The purified exopolysaccharide of Bacillus sp. SGD-03 was applied to ID and 2D NMR analysis to elucidate the exact structure of a polysaccharide. The 20.0 mg powder of EPS was dissolved in 0.6 mL of D₂O and kept overnight at 4° C. for proper disintegration. Spectra of ID and 2D including 1 H (500.13 MHZ), 13C (125.76 MHz), DEPT, NOESY, COSY, HSQC, and HMBC, were recorded with Bruker Avance spectrometer 500. All chemical shifts (δ) are reported in parts per million downfield from D₂O (4.71 ppm), as an internal standard. Spin multiplets are reported as d (doublet) and t (triplet). Coupling constant (J) is reported in hertz (Hz).

To determine the glycosidic linkage pattern between the sugar residues and elucidation of structure the spectra of ¹D and ²D ¹H and ¹³C NMR were analyzed. The ¹H NMR seven signal at the range of δ 3.48 and δ 4.12 (δ 3.48, δ 3.60, δ 3.71, δ 3.83, δ 3.89, δ 4.03, and δ 4.12) and ¹³C NMR shows six signals at a range of δ 59.9 to δ 80.29 (δ 59.9, δ 63.4, δ 75.2, δ76.3, 80.29) shows characteristics peaks of sugar. Also in 13C NMR spectra signal at δ 104.2 shows the characteristics peaks of anomeric carbon present in the sugar). The DEPT and HSQC spectra show two methylene peaks at δ 59.9 (δ 3.71 and 3.60) and δ 63.4 (δ 3.48 and δ 3.83), indicating the presences of the keto-sugars. In the COSY spectra, the signal at δ 3.48 and δ 3.83 shows a correlation of δ 3.89 protons. The proton at δ 3.89 correlations at δ 4.03 proton and further this δ 4.03 proton shoes correlation with δ 4.12 proton indicate that both are serially connected. After careful analysis of ID and 2D NMR and literature report, confirmed the fructose sugar (Taylan, O., et al (2019). Partial characterization of a levan type exopolysaccharide (EPS) produced by Leuconostoc mesenteroides showing immunostimulatory and antioxidant activities. International Journal of Biological Macromolecules, 136, 436-444, https://doi.org/10.1016/j.ijbiomac.2019.06.078). The linkage of the fructose sugar was confirmed using NOESY NMR spectra. The proton on C-3 carbon δ 4.13 shows NOESY correlation peak at proton present on C⁻¹ carbon of δ 3.71 and δ3.61 indicating that both are the same plane, hence the sugar is joined by beta linkage. The chemical shift of carbon was also compared with previously reported levan from other sources. All the NMR spectra confirm that the purified EPS is Levan which is linked by β (2-6)-D-fructofuranosyl residues (Han, Y. W., & Clarke, M. A. (1990). Production and characterization of microbial levan. Journal of Agricultural and Food Chemistry, 38(2), 393-396 Pei, F., Ma, Y., Chen, X., & Liu, H. (2020). Purification and structural characterization and antioxidant activity of levan from Bacillus megaterium PFY-147, International Journal of Biological Macromolecules, 161, 1181-1188. https://doi.org/10.1016/j.ijbiomac.2020.06 140)

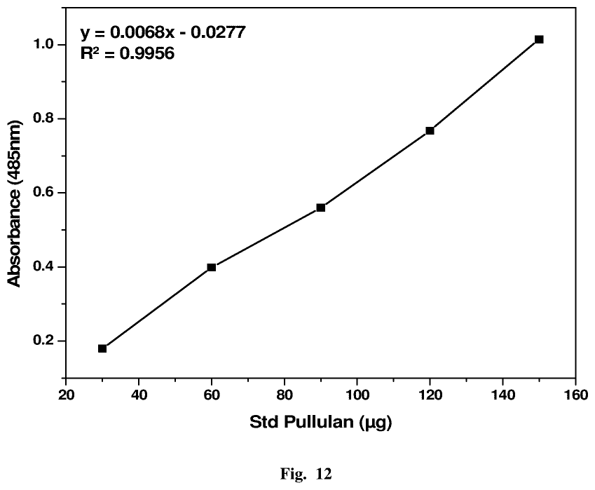

The biomass concentration was calculated by diluting the known concentration of dry weight of the cell as per the requirement. The value of readings of optical density at 600 nm and light scattering at 620 nm was plotted and biomass concentration was calculated using regression equation y=0.0502x+0.0106 with R² value 0.9936. All the EPS quantification was done by revaluated phenol sulphuric acid method by (Rao, P., & Pattabiraman, T. N. (1989). Reevaluation of the phenol-sulfuric acid reaction for the estimation of hexoses and pentoses, Analytical biochemistry, 181(1), 18-22, https://doi.org/10.1016/0003-2697(89)90387-4). The unknown concentration of EPS was estimated by a linear graph of standard pullulan polysaccharides (FIG. 12).

TABLE 3
Cholesterol reduction analysis in animal studies

| | | Total | | LDL- | HDL- |
|---|---|---|---|---|---|
| | Glucose | Cholesterol | Triglycerides | Cholesterol | Cholesterol |
| Groups | (mg/dl) | (mg/dl) | (mg/dl) | (mg/dl) | (mg/dl) |
| Group-I | 85.17 ± 2.05 | 63.25 ± 2.40 | 60.14 ± 2.55 | 73.76 ± 7.19 | 7.52 ± 0.59 |
| (Vehicle | | | | | |
| control) | | | | | |
| Group-II | 88.83 ± 2.53 | 71.57 ± 4.61 | 87.55 ± 2.21*** | 80.92 ± 5.93 | 8.2 ± 0.75 |
| (Standard | | | | | |
| control) | | | | | |
| Group-III | 96.0 ± 2.14 | 62.41 ± 2.20 | 77.37 ± 1.04*** | 70.32 ± 5.86 | 8.72 ± 0.39 |
| (100 mg/kg | | | | | |
| of test | | | | | |
| compound) | | | | | |
| Group-IV | 92.0 ± 3.41 | 63.62 ± 4.16 | 87.64 ± 1.92*** | 84.40 ± 2.38 | 7.48 ± 0.74 |
| (250 mg/kg | | | | | |
| of test | | | | | |
| compound | | | | | |
| Group-V | 91.0 ± 2.88 | 61.25 ± 1.99 | 91.75 ± 1.03*** | 76.71 ± 7.52 | 9.96 ± 0.39 |
| (500 mg/kg | | | | | |
| of test | | | | | |
| compound) | | | | | |

Note:
Values are expressed as Mean ± SEM, n = 6, Values of standard control and test compound treated groups are compared with Vehicle control by ANOVA followed by Dunnett's test. Software Used for data analysis: GraphPad Prism 9.2 version
***p < 0.0001 when compared with normal control

TABLE 4
Comparison with EPS yield with related arts

| SI. | | | Fermentation | Production | | Levan | | Quantification | | |
|---|---|---|---|---|---|---|---|---|---|---|
| No. | Organisms | Substrate | Type | duration | | quantity | | methodology | Reference | Source |
| 1 | Bacillus | Sucrose | SmF | 10 | days | 40 | g/L | Ethanol | Han | Soil |
| | polymyxa | (8%) | | | | | | Precipitate | (1989) | |
| | | | | | | | | EPS | | |
| 2 | Zymomonas | Sucrose | SmF | 72 | h | 14.67 | g/L | Ethanol | Melo, et | Culture |
| | mobilis | (25%) | | | | | | Precipitate | al., (2007 | collection |
| | | Yeast | | | | | | EPS | | |
| | | extract | | | | | | | | |
| 3 | Bacillus | Sucrose | SmF | 48 | h | 18.0 | g/L | Ethanol | Arab, | Culture |
| | licheniformis | (0.15%) | | | | | | Precipitate | Mahmoud, | collection |
| | | | | | | | | and dialyzed | and Higgins | |
| | | | | | | | | EPS | (2007 | |
| 4 | Pseudomonas | Sucrose | SmF | 6 | days | 15.42 | g/L | Ethanol | Jathore | Culture |
| | fluorescens | (6%) | | | | | | Precipitate | et al. | collection |
| | | | | | | | | EPS | (2012) | |
| 5 | Gluconacetobacter | Sucrose | SmF | 34 | h | 8.29 | g/L | Ethanol | Molinari | Culture |
| | diazotrophicus | (10%) | | | | | | Precipitate | and | collection |
| | | | | | | | | and Dialysed | Boiardi | |
| | | | | | | | | EPS | (2013 | |
| 6 | Bacillus | Sucrose | SmF | 36 | h | 30.0 | g/L | Ethanol | Dahech | Thermal |
| | licheniformis | (5%) | | | | | | Precipitate | et al. | source |
| | | | | | | | | Dialysed EPS | 2013 | |
| 7 | Zymomonas | Sucrose | SmF | 42.3 | h | 40.2 | g/L | Ethanol | Silbir, | Culture |
| | mobilis | (15-30%) | | | | | | Precipitate | et al., | collection |
| | B-14023 | | | | | | | EPS | (2014) | |
| 8 | Bacillus | Mango | SSF | 72 | h | 15.6 | g/L | Ethanol | Asgher | Mutated |
| | licheniformis | peels | | | | | | Precipitate | et al., | strain |
| | | | | | | | | EPS | 2019 | |
| * | Bacillus | Sucrose | SmF | 21 | h | 123.9 | g/L | Ethanol | Current | Marine |
| | licheniformis | (4%) | | | | | | Precipitate | study | isolate |
| | SGD-03 | | | | | | | EPS | | |

SmF: Submerged fermentation

### Advantages of the Invention

High yield production of Levan-EPS from marine bacteria.
Increased production of levan free of contaminating polysaccharides.
Cost-effective process of preparing Levan-EPS.
Production of Levan-EPS within 24 hr.
Production of Levan-EPS with cholesterol-lowering properties.
The production of levan is in well-controlled conditions, without the intervention of different times (day), less manpower, and 48 fermentation run on single plate form could be achieved with the help of a micro bioreactor.
Thus, the present process covers media utilization in less time (21 h) with cost-effective media formulation, and that to, a low amount of media with complete utilization and improved production in a short time displays the robustness of the process.
In nutshell, the yield of levan was obtained around 107-123 g/L by supplying 40 g/L of sucrose, using marine organisms wherein the levan has uniform MW of 10 kDa, which has been never reported till date in the literature irrespective of different source of microorganism.

I/We claim:

1. A process for production of a levan essentially free of a contaminating polysaccharide using a micro-bioreactor, comprising the steps of:
a) preparing and optimizing a media comprising 4 to 10% of sucrose using a microtiter plate in a micro-bioreactor;
b) inoculating a marine Bacillus sp. in the media and incubating at 28° C. for 18-24 hours;
c) centrifuging the incubated media at 10000 rpm to obtain a cell-free supernatant;
d) precipitating the cell free supernatant using an absolute ethanol solvent to obtain precipitated exopolysaccharide (EPS);
e) separating the precipitated EPS in absolute ethanol by centrifugation followed by washing with a solvent;
f) dissolving washed EPS of step e) in a solvent to make a solution containing levan; and
g) lyophilizing the solution containing levan.

2. The process as claimed in claim 1, wherein the marine Bacillus sp. is SGD-03 having accession no. MCC 0243.

3. The process as claimed in claim 1, wherein the yield of the levan is in range of 107 to 123 g/It with 99% of purity.

4. The process as claimed in claim 1, wherein molecular weight of the levan is in the range of 1.0×10⁴ kDa.

5. The process as claimed in claim 1, wherein the media is a composition of polysaccharides, peptone, beef extract and salt.

6. The process as claimed in claim 5, wherein the media comprises of 0.4% of peptone, 0.4% of beef extract, 0.5% of NaCl, 4% of sucrose and water.

7. The process as claimed in claim 1, wherein the marine Bacillus sp. is grown for 8-14 hrs in a rotatory shaker at 150 rpm for 12-18 h to prepare the inoculum.

8. The process as claimed in claim 1, wherein the solvent for precipitation in step (d) is three-volume of pre-chilled absolute ethanol (4° C.) and the solvent for washing in step e) is 70% ethanol.

9. The process as claimed in claim 1, wherein the EPS is dissolved using solvent Milli Q H₂O.

10. The process as claimed in claim 1, wherein diameter of levan fibers obtained is in the range of 3.620 μm to 9.071 μm.
